(19) United States
(12) Patent Application Publication
(10) Pub. No.: US 2021/0169403 A1
(43) Pub. Date: Jun. 10, 2021

(54) METHOD OF DETECTING CRACKS IN TEETH

(71) Applicant: GreenMark Biomedical Inc., East Lansing, MI (US)

(72) Inventors: Steven BLOEMBERGEN, Okemos, MI (US); Nathan A. JONES, Ann Arbor, MI (US)

(21) Appl. No.: 17/096,586

(22) Filed: Nov. 12, 2020

Related U.S. Application Data
(60) Provisional application No. 62/944,487, filed on Dec. 6, 2019.

(51) Int. Cl. A61B 5/00

(52) U.S. Cl. CPC A61B 5/4547; A61B 5/0088; A61B 5/0077

(57) ABSTRACT

Cracks in teeth can be detected and/or diagnosed by applying cationic fluorescent nanoparticles to the teeth. Optionally, the teeth are first cleaned to remove substances such as plaque. The nanoparticles can then be applied, for example by dispersing them in a mouthwash or gel that is applied to the teeth. The teeth are then rinsed, for example with water, to remove nanoparticles that are not adhered to the teeth. The teeth are then exposed to light in the excitation range of the nanoparticles. The teeth are then observed, either by eye or with a sensor or imaging device, to determine the presence of fluorescent light in the shape of a crack, which may be for example a line.

### CROSS-REFERENCE TO RELATED APPLICATIONS

This application claims the benefit of U.S. Application Ser. No. 62/944,487, filed Dec. 6, 2019, which is incorporated herein by reference.

### FIELD

This specification relates to dental diagnostic methods, and in particular to methods of detecting cracks in teeth.

### BACKGROUND

Cracks extend though the enamel of a tooth into the dentin, whereas craze lines only affect the enamel. If left untreated, cracks may enlarge into the pulp or periodontal ligament. Cracks are often associated with caries, but about one third of cracks appear in teeth that are free of caries. The presence of a crack can be initially indicated by pain or other symptoms. However, determining the depth and orientation of the crack is difficult since cracks do not often appear in radiographs. Some cracks are visible, either unaided or with magnifying loupes or trans-illumination. Some cracks can be located by dental probes or bite tests, although these methods can be painful and risk propagating the crack. Stains or dyes such as gentian violet or methylene blue have also been used to highlight cracks in teeth.

### INTRODUCTION

The following introduction is intended to introduce the reader to the detailed description to follow and not to limit or define any claimed invention.

This specification describes a method of detecting a crack in a tooth. A crack is detected by applying fluorescent nanoparticles to one or more teeth. Optionally, the teeth are first cleaned to remove substances such as plaque that might also attract the nanoparticles or physically prevent their entry into the crack. The nanoparticles can then be applied, for example by dispersing them in a mouthwash that is swished in the mouth or a gel that is applied to the teeth. Optionally, the teeth are then rinsed, for example with water, to remove nanoparticles that are not adhered to the teeth. The teeth are then exposed to light in the excitation range of the nanoparticles. The teeth are then observed, either by eye or with a sensor or imaging device such as a camera, in either case with magnification or not, to determine the presence of fluorescent light in the shape of a crack. The fluorescent nanoparticles may break down in saliva. Remains of the fluorescent nanoparticles may be spit out of the mouth, optionally with the aid of a water rinse. In some examples, the fluorescent nanoparticles are cationic, for example having a positive zeta potential at a neutral pH. In some examples, the fluorescent nanoparticles are starch based. In some examples, the fluorescent nanoparticles comprise fluorescein, which can be illuminated with a dental curing lamp or other blue light.

This specification also describes the use of fluorescent nanoparticles to detect a crack in a tooth; fluorescent nanoparticles for use as an agent for the detection of a crack in a tooth; and, fluorescent nanoparticles for use in detecting a crack in a tooth. In some examples, the fluorescent nanoparticles are part of an aqueous composition such as a mouth rinse or gel. In some examples, the fluorescent nanoparticles are cationic, for example having a positive zeta potential at a neutral pH. In some examples, the fluorescent particles nanoparticles are starch based. In some examples, the fluorescent nanoparticles comprise fluorescein, which can be illuminated with a dental curing lamp or other blue light.

### BRIEF DESCRIPTION OF FIGURES

FIG. 1 shows an extracted tooth with a demineralized area that has been treated with fluorescent nanoparticles, illuminated under blue light and photographed through an orange filter.

FIG. 2 shows a sliver of an extracted tooth with a demineralized area in the lower two-thirds of the sliver, photographed through a stereo-microscope, illuminated in the center and right panels under blue light and photographed through an orange filter.

FIG. 3 shows a sliver of an extracted tooth with a demineralized area, in the lower panel photographed through a stereo-microscope illuminated in the lower panel under blue light and photographed through an orange filter, and in the upper panel a micro-CT scan.

FIG. 4 shows an extracted tooth with an occlusal lesion and cracks that has been treated with fluorescent nanoparticles, in the right hand panels illuminated under blue light and photographed through an orange filter.

### DETAILED DESCRIPTION

International Publication Number WO 2017/070578 A1, Detection and Treatment of Caries and Microcavities with Nanoparticles, published on Apr. 27, 2017, describes nanoparticles for detecting microcavities in teeth. The nanoparticle comprises a biopolymer such as starch and a fluorescent agent. The nanoparticles have a net positive charge, as determined by a positive zeta potential at neutral pH, and associate with microcavities. The location of microcavities can be indicated, for example, by fluroescence of the nanoparticles under the influence of blue light, such as a dental curing lamp. International Publication Number WO 2017/070578 A1 is incorporated herein by this reference to it.

International Publication Number WO 2019/191456 A1, Phosphate Crosslinked Starch Nanoparticles and Dental Treatments, published on Oct. 3, 2019, describes starch nanoparticles made using an emulsion process with a phosphorous compound such as STMP as a crosslinking agent. Negative change of the nanoparticles can be reduced or reversed by adding cations or cationizing the starch or both. In some examples, fluorescein is added. In some examples, the nanoparticles have a postive zeta potential at neutral pH. In some examples, the nanoparticles have a positive zeta potential at pH of 5.5 and under and a negative zeta potential at pH of 7 and above. International Publication Number WO 2019/191456 A1 is incorporated herein by this reference to it.

In International Publication Number WO 2017/070578 A1, it is stated that active carious legions have a negative charge. The negative charge may be caused by the release of cationic ions during demineralization, resulting in the remaining demineralized enamel having a negative charge. Positively charged nanoparticles may be attracted to the negatively charged demineralized areas. Cracks in teeth might or might not be demineralized. In cases where cracks pass through or extend from a carious lesion, the inventors expect that the crack may also be demineralized. However, a recent crack in a tooth without caries might not be demineralized.

The word cracks includes cracks or fractures in any orientation or location, including oblique cracks and vertical cracks. An oblique crack may be part of, or located so as to develop into, a fractured cusp. A vertical crack may be an incomplete fracture initiated from the crown and extending subgingivally. In other cases, a vertical crack may be part of a split tooth.

The word nanoparticles is used herein according to common usage in North America, and may include particles up to 1000 nm in size. In some countries, these particles will be called micro-particles or submicron particles. The nanoparticles may also meet the IUPAC definition of a nanoparticle and be less than 100 nm in size, but this is optional. Unless stated otherwise, the size of a nanoparticle is its z-average size as determined by dynamic light scattering.

Nanoparticles as described in International Publication Number WO 2017/070578 A1 or International Publication Number WO 2019/191456 A1 may be used to detect cracks. The nanoparticles optionally have a positive zeta potential at neutral pH. Optionally, the nanoparticles have a positive zeta potential at pH of 5.5 and under and a negative zeta potential at pH of 7 and above. The nanoparticles may be starch based. The nanoparticles may be dispersed into water to form a mouth rinse that can be swished around in the mouth of a person. In this way the nanoparticles are applied to many teeth simultaneously. Alternatively, the nanoparticles may be carried in a gel and applied to a particular tooth of interest.

In examples described herein, starch based cationic nanoparticles were made as described in International Publication Number WO 2017/070578 A1, Detection and Treatment of Caries and Microcavities with Nanoparticles. The nanoparticles are 30 to 250 nm in size. The starch in the nanoparticles is functionalized with fluorescein. Fluorescein gives off green light when excited by blue light, for example light from a dental curing lamp. The fluorescent response is visible to the eye of a person wearing UV protective glasses, for example orange tinted glasses. These glasses are typically long pass filters. A filter may have a cut in frequency in the range of about 520-550 nm. The fluorescent response can also be captured by a camera or CMOS or CCD sensor through a similar filter. Optionally the image can be further manipulated, for example by selecting a green pixel only image using Image J or other image analysis software.

In various examples, the nanoparticles were dispersed in water. Cleaned human extracted teeth, or portions of extracted teeth, where immersed in the nanoparticle dispersion typically for about 30 seconds. The extracted teeth were removed from the nanoparticle dispersion and immersed in water for rinsing.

Each of the rinsed teeth were then placed below a camera or other sensor. An orange plastic filter was placed between the camera and the tooth. The filter is of the type used to protect dental professionals from blue light exposure and therefore models visual observation of the tooth. A blue light dental curing lamp was shined against the teeth. Photographic images were taken of the tooth.

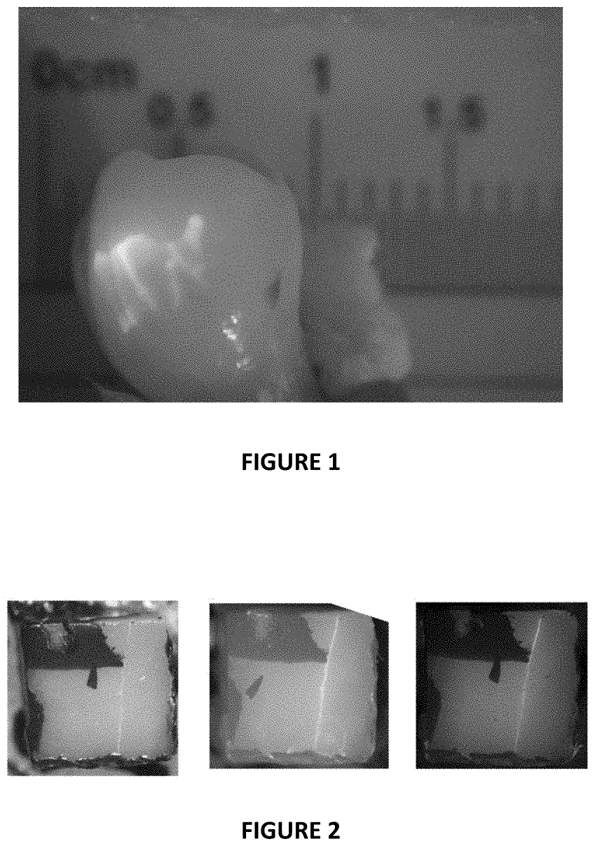

In one example, an area on the side of an extracted tooth was artificially demineralized. As shown in FIG. 1, the roughly rectangular demineralized area is mildly fluorescent. However, various bright lines of fluorescence are visible in the demineralized area. Very faint extensions of these bright lines are also visible extending from the demineralized area in the original image, although these faint lines might be difficult or impossible to see in the reproduction herein. Although the presence of these cracks or craze lines in the tooth was not previously known, since the bright lines do not correspond with the demineralization pattern they are believed to indicate the presence of cracks or craze lines. The inventors believe that the cracks or craze lines may have formed as a result of the extracted tooth drying out. It is also possible that craze lines or cracks in the tooth were widened by the demineralization treatment in the area of the demineralization treatment. It is expected that even if the features illuminated by the nanoparticles are craze lines, the nanoparticles would also illuminate a crack.

The exact mechanism by which the nanoparticles aggregate in the craze lines or cracks is not known. There may be physical entrapment of the nanoparticles in the cracks. This appears to be a possible mechanism in particular for the parts of the craze lines or cracks that extend beyond the demineralized area, which are not expected to be negatively charged. Within the demineralized area, physical entrapment is still possible. It is also possible that the cracks or craze lines in this area become negatively charged when this area was demineralized after the cracks or craze lines formed. Accordingly, there might be physical entrapment of the nanoparticles, charge attraction of the crack or craze lines to the nanoparticles, or both.

It is expected that the nanoparticles described herein will detect naturally formed cracks or craze lines, including vertical cracks and fractured cusps. Image filtering may be used to provide enhanced images of the crack.

Without intending to be limited by theory, it is possible that the nanoparticles become physically trapped in a crack, are charge attracted to a crack, or are charge or otherwise attracted to dentin. Alternatively, cracks may have a negative charge or develop a negative charge soon after a crack is formed. Optionally, the nanoparticles may have a positive charge, for example a positive zeta potential at neutral pH. Optionally, the nanoparticles have another charge. The nanoparticles may be administered to the mouth of a patient in an aqueous form, for example a dispersion of the nanoparticles in a mouth rinse or topically applied gel.

In another example, a sliver was taken from an extracted tooth. The sliver includes enamel and dentin. The top third of the sliver was covered with a red polish and the bottom two thirds of the sliver was left exposed. A mild demineralizing treatment was then applied to the tooth. About one half of the red polish was scraped away after the demineralization treatment was completed. The sliver was soaked for 30 seconds in a dispersion of the nanoparticles and then rinsed in water. Referring to the left hand panel of FIG. 2, a crack or craze line is visible under normal light through a stero-microscope. In the middle panel, an orange filter is placed between the stereo-microscope and the sliver and the sliver is illuminated with blue light. The top third of the tooth was originally all covered in the red polish and so was not demineralised. A generally vertical crack or craze line is visible extending through the demineralised area and through the non-demineralised area of the tooth. In the right hand panel, only the green pixels of the camera are selected. This example confirms that detection of a crack or craze line does not depend on the crack or craze line being demineralized.

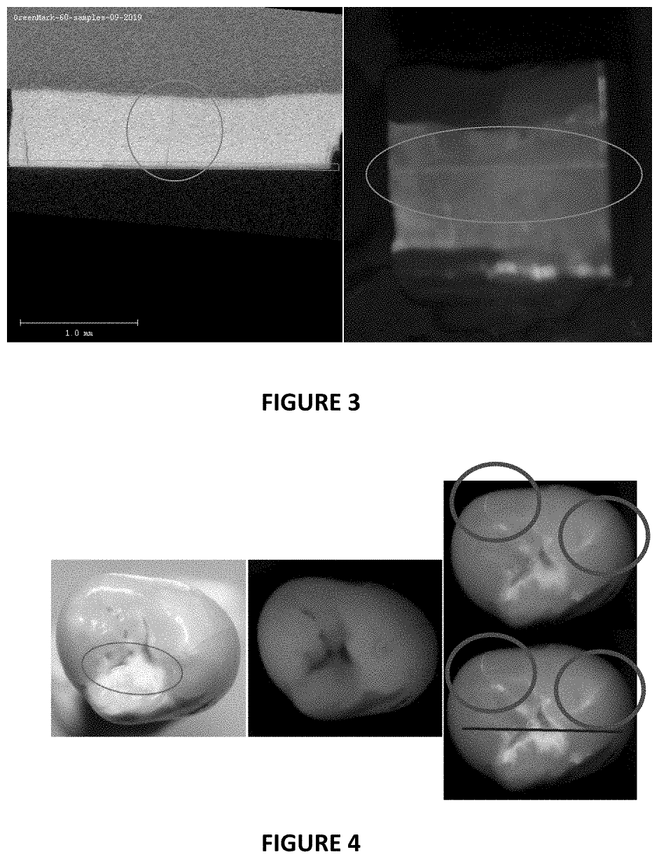

In another example, a sliver was taken from an extracted tooth. The sliver includes enamel and dentin. Sections at the top and bottom of the sliver were covered with a red polish and the middle of the sliver was left exposed. A demineralizing treatment was then applied to the tooth. The sliver was soaked for 30 seconds in a dispersion of the nanoparticles and then rinsed in water. Referring to the bottom panel of FIG. 3, an orange filter is placed between a stereo-microscope and the sliver and the sliver is illuminated with blue light. A crack or craze line is visible extending horizontally across the tooth even against the background fluorescence of the demineralized area. In the top panel, a micro-CT scan shows a cross-sectional slice of the sliver. The crack or craze line, circled in blue in the lower panel, is also circled in blue in the upper panel. The crack or craze line extends completely though the enamel (white area) and into the grey area (dentin). Despite this extension to the dentin, it is not clear whether the feature is a crack or a craze line. Although craze lines are typically described as not extending to dentin, it may be that craze lines do at least sometimes extend to dentin. If craze lines do extend to dentin, the distinction between cracks and craze lines might be that craze lines are not strongly associated with dentinal sensitivity, that craze lines are not oriented in a way that causes them to be strained by biting, and/or that craze lines are not likely to propagate. The distinction between a crack that needs to be treated and a craze line that does not require treatment might therefore be made based on the presence or absence of symptoms such as pain or sensitivity and the location, length and/or orientation of the crack or craze line and/or whether the crack or craze line is associated with a carie.

FIG. 4 shows an extracted tooth with an occlusal lesion. The tooth was soaked for 30 seconds in a dispersion of the nanoparticles and then rinsed in water. In the right hand panel, the tooth was illuminated with blue light and photographed through an orange filter, with and without green pixel selection. As indicated in the circled areas, cracks extend from the lesion and are illuminated by the nanoparticles. These are believed to be cracks because of their characteristic location extending from a carie in the biting surface of the tooth.

In the examples described herein, the teeth were treated using nanoparticles as described in International Publication Number WO 2017/070578 A1. Alternatively or additionally, nanoparticles as described in International Publication Number WO 2019/191456 A1 may be used. These phosphorous containing nanoparticles may be advantageous. Since they tend to remineralize carious lesions and reduce dentinal sensitivity, they might also restore a crack or craze line. The phosphorous containing nanoparticles might optionally be used without fluorescein in them as a restorative treatment rather than to detect a crack or craze line.

We claim:

1. A method of detecting, locating, viewing, or diagnosing a crack or craze line in a tooth comprising the steps of,
applying fluorescent nanoparticles to the tooth;
applying light in the excitation range of the fluorescent nanoparticles to the tooth; and,
observing the presence of a fluorescent response from the tooth in an elongated pattern.

2. The method of claim 1 comprising cleaning the tooth before applying fluorescent nanoparticles to the tooth.

3. The method of claim 1 wherein the nanoparticles are cationic.

4. The method of claim 1 wherein the nanoparticles are dispersed in an aqueous liquid or a gel.

5. The method of claim 1 comprising rinsing the tooth after applying the fluorescent nanoparticles to the tooth.

6. The method of claim 1 wherein the fluorescent response is observed by eye, optionally through a protective filter.

7. The method of claim 1 wherein the tooth is observed with a sensor, camera or other imaging device.

8. The use of fluorescent nanoparticles to detect a crack or craze line in a tooth;
fluorescent nanoparticles for use as an agent for the detection of a crack or craze line in a tooth; or, fluorescent nanoparticles for use in detecting a crack or craze line in a tooth.
